(19) United States
(12) Patent Application Publication
(10) Pub. No.: US 2013/0261161 A1
(43) Pub. Date: Oct. 3, 2013

(54) ANTIPARASITIC FORMULATIONS

(71) Applicant: VELCERA, INC., Yardley, PA (US)

(72) Inventors: John PRESTON, Yardley, PA (US); David M. Petrick, Princeton, NJ (US)

(73) Assignee: Velcera, Inc., Yardley, PA (US)

(21) Appl. No.: 13/804,264

(22) Filed: Mar. 14, 2013

Related U.S. Application Data
(63) Continuation-in-part of application No. 13/632,919, filed on Oct. 1, 2012.
(60) Provisional application No. 61/541,987, filed on Sep. 30, 2011.

(51) Int. Cl. A61K 31/415; A61K 31/231

(52) U.S. Cl. CPC A61K 31/415; A61K 31/231
USPC 514/404

(57) ABSTRACT

Fipronil co-formulations are provided herein. The formulations comprise an organic solvent, an alcohol co-solvent, and one or more antioxidants. The formulations provided herein are antiparasitic, and can be used, for example, to combat dog and cat parasites, such as, fleas and ticks.

### CROSS-REFERENCE TO RELATED APPLICATION

This application is a continuation-in-part of U.S. application Ser. No. 13/632,919, filed Oct. 1, 2012, which claims the benefit of priority to U.S. Provisional Application No. 61/541,987, filed on Sep. 30, 2011 and entitled “Antiparasitic Formulations”, the contents of each of which are hereby incorporated by reference in their entireties for all purposes.

### FIELD OF THE INVENTION

The present invention relates to an antiparasitic formulation for treatment and protection of animals.

### BACKGROUND OF THE INVENTION

Animals are commonly infested with parasites, for example, fleas (e.g., Ctenocephalides felis, Ctenocephalides sp.), ticks (e.g., Rhipicephalus sp., Ixodes sp., Dermacentor sp., Amblyomma sp.), and mites (e.g., Demodex sp., Sarcoptes sp., Otodectes sp).

Fleas cause an animal a great deal of stress and are harmful to the animal's health. Moreover, fleas are also vectors of pathogenic agents, such as dog tapeworm (Dipylidium caninum), and can also attack man. Similarly, ticks can also cause an animal stress and be harmful to its health.

The goals when formulating an antiparasitic treatment should include both quick knock down of fleas already present on animals followed by a long duration of action in preventing further infestations. Chemicals of the pyrethroid family, such as permethrin, are able to achieve quick killing of fleas and ticks while agents such as fipronil and s-methoprene demonstrate longer term efficacy in treating and preventing future infestations. Typically, spot-on formulations which contain permethrin contain more than about 40% w/w of the pyrethroid. However, in developing a triple combination product such as the products described herein, it is advantageous to decrease the percentage of permethrin. The principal challenges in developing a product such as this is reducing the volume of fluid to a convenient volume without losing the benefit of the permethrin-mediated quicker knock down or the translocation abilities of the other actives agents and solvent systems.

The present invention provides antiparasitic formulations to treat parasite infestations of animals.

### SUMMARY OF THE INVENTION

In one embodiment, a fipronil formulation is provided comprising fipronil or a veterinary acceptable salt thereof, which is about 9% to about 11% by weight of the formulation; at least one C₁-C₆ alcohol co-solvent, wherein the total amount of C₁-C₆ alcohol co-solvent is up to about 5% by weight of the formulation; one or more antioxidants, wherein the total amount of the one or more antioxidants are about 0.005% to about 1.0% by weight of the formulation; at least one organic solvent which is one or more glycol ethers selected from the group consisting of diethylene glycol monoethyl ether, ethylene glycol monoethyl ether, dipropylene glycol n-butyl ether, dipropylene glycol monomethyl ether, and combinations thereof, wherein the total amount of the at least one organic solvent makes up the balance of the formulation; and the formulation does not contain a surfactant or a crystallization inhibitor.

In another embodiment, a co-formulation is provided comprising fipronil or a veterinary acceptable salt thereof, which is about 9% to about 11% by weight of the formulation; S-methoprene, or a veterinary acceptable salt thereof; at least one C₁-C₆ alcohol co-solvent, wherein the total amount of C₁-C₆ alcohol co-solvent is up to about 5% by weight of the formulation; one or more antioxidants, wherein the total amount of the one or more antioxidants are about 0.005% to about 1.0% by weight of the formulation; at least one organic solvent which is one or more glycol ethers selected from the group consisting of diethylene glycol monoethyl ether, ethylene glycol monoethyl ether, dipropylene glycol n-butyl ether, dipropylene glycol monomethyl ether, and combinations thereof, wherein the total amount of the at least one organic solvent makes up the balance of the formulation; and the formulation does not contain a surfactant or a crystallization inhibitor.

In one embodiment, a spot-on formulation is provided comprising fipronil or a veterinary acceptable salt thereof which is present in an amount of about 5 to about 15% by weight of the formulation, an insect growth regulator in an amount of about 7 to about 12% by weight of the formulation, a pyrethroid in an amount of about less than about 40% by weight of the formulation and an organic solvent. In one embodiment, the insect growth regulator is s-methoprene and the pyrethroid is permethrin. In one embodiment, the formulation also contains a potentiator of permethrin. In one embodiment, the potentiator of permethrin is piperonyl butoxide.

In one embodiment, a spot-on formulation is provided comprising fipronil or a veterinary acceptable salt thereof which is present in an amount of about 5 to about 15% by weight of the formulation, an insect growth regulator in an amount of about 7 to about 12% by weight of the formulation, a pyrethroid in an amount of about less than 20% by weight of the formulation and an organic solvent. In one embodiment, the insect growth regulator is s-methoprene and the pyrethroid is permethrin. In one embodiment, the formulation also contains a potentiator of permethrin. In one embodiment, the potentiator of permethrin is piperonyl butoxide.

In one embodiment, a spot-on formulation is provided comprising fipronil or a veterinary acceptable salt thereof which is present in an amount of about 5 to about 15% by weight of the formulation, a juvenile hormone analogue in an amount of about 1 to about 10% by weight of the formulation, a pyrethroid in an amount of about less than about 40% by weight of the formulation and an organic solvent. In one embodiment, the juvenile hormone analogue is pyriproxifen and the pyrethroid is permethrin. In one embodiment, the formulation also contains a potentiator of permethrin. In one embodiment, the potentiator of permethrin is piperonyl butoxide.

In one embodiment, a spot-on formulation is provided comprising fipronil or a veterinary acceptable salt thereof which is present in an amount of about 5 to about 15% by weight of the formulation, a juvenile hormone analogue in an amount of about 1 to about 10% by weight of the formulation, a pyrethroid in an amount of about less than 20% by weight of the formulation and an organic solvent. In one embodiment, the juvenile hormone analogue is pyriproxifen and the pyrethroid is permethrin. In one embodiment, the formulation also contains a potentiator of permethrin. In one embodiment, the potentiator of permethrin is piperonyl butoxide.

In one embodiment, the spot-on formulation comprises a potentiator of permethrin. In some embodiments the potentiator of permethrin is piperonyl butoxide. In one embodiment, piperonyl butoxide is present in an amount of about 0.1% to about 20% w/w of the formulation. In another embodiment, piperonyl butoxide is present in an amount of about 0.5% to about 15% w/w of the formulation. In another embodiment, piperonyl butoxide is present in an amount of about 1.0% to about 10% w/w of the formulation. In another embodiment, piperonyl butoxide is present in an amount of about 3% to about 8% w/w of the formulation. In another embodiment, piperonyl butoxide is present in an amount of about 2% to about 5% w/w of the formulation. In another embodiment, piperonyl butoxide is present in an amount of about 10% to about 20% w/w of the formulation. In another embodiment, piperonyl butoxide is present in an amount of about 5% to about 15% w/w of the formulation.

In one embodiment, the spot-on formulation comprises a C₁-C₆ alcohol co-solvent. In one embodiment, the C₁-C₆ alcohol co-solvent is ethanol, propanol, isopropanol, or a combination thereof. In one embodiment, the C₁-C₆ alcohol co-solvent is ethanol. In another embodiment, the C₁-C₆ alcohol co-solvent is isopropanol. In yet another embodiment, C₁-C₆ alcohol co-solvent is a combination of ethanol and isopropanol.

In one embodiment, the spot on formulation comprises one or more antioxidants. In one embodiment, the one or more antioxidant is butylated hydroxianisole (BHA), butylated hydroxytoluene (BHT) and/or α-tocopherol.

In one embodiment, the organic solvent is diethylene glycol monoethyl ether.

In one embodiment, the spot-on formulation is effective, when applied to a mammal, in killing about 90% or more of fleas within 3 hours of application. In another embodiment, the spot-on formulation is effective, when applied to a mammal, in killing more than about 90% of fleas within 3 hours of application. In one embodiment, the mammal is a dog. In another embodiment, the mammal is a cat.

### DETAILED DESCRIPTION OF THE INVENTION

Various embodiments and advantages of the present invention will be set forth in part in the description that follows, and in part will be obvious from the description, or may be learned by practice of the invention. It is to be understood that both the foregoing general description and the following detailed description are exemplary and explanatory only and are not restrictive of the invention as described.

The terms “a” and “an” do not denote a limitation of quantity, but rather denote the presence of at least one of the referenced item. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

The present invention is directed to veterinary formulations comprising fipronil and co-formulations of fipronil and S-methoprene. In one embodiment, the formulations of the invention are administered topically. For example, the formulation can be provided as a dispersion, solution, emulsion, suspension, ointment, cream, paste, gel or lotion. In one embodiment, the formulation of the invention is a “spot-on” formulation.

Spot-on formulations are applied by local point application to the animal. More specifically, spot-on formulations may be applied to animals by deposition on the skin; this may be a localized application in particular at one, two, or more points and preferably localized between the animal's shoulders and along the mid-line of the back. After deposition, the formulation dries and diffuses over the animal's entire body without crystallizing or changing the appearance (in particular absence of any whitish deposit or of any dusty appearance) or the feel of the coat. The formulation is typically applied over a surface area of up to 10 cm², normally from 5 and 10 cm².

In a spot-on formulation, the alcohol co-solvent is the drying agent or drying promoter; while a surfactant and/or a crystallization inhibitor can improve the stability of the formulation, for example, by preventing crystallization of the active ingredient(s). A spot-on formulation is applied on a small, localized area of an animal, after which it dries and diffuses over the animal's entire body.

To achieve the desirable efficacy, the conventional spot-on formulations use surfactant(s)/crystallization inhibitor(s) and/or a certain amount of alcohol to assure that, when applied locally, the formulation can effectively dry and spread over the animal's entire body. For example, the FRONTLINE® products from Merial, Inc. contains about 10% ethanol and crystallization inhibitor(s).

It has been surprisingly found that the present spot-on formulations which contain very low alcohol content, e.g., about 5% or below, and are without any surfactant or crystallization inhibitor, retain the desirable efficacy, while at the same time, after drying, give good appearance and feel of non-sticky coat after application. In other words, despite the very low alcohol content and the absence of the surfactant and crystallization inhibitor, the present spot-on formulations, when applied to an animal locally, subsequently dry and spread over the animal's entire body, while at the same time avoiding any phenomenon of crystallization over a significant time period.

Additionally, it has been surprisingly found that the present spot-on formulations which contain less than 40% w/w of a pyrethroid, such as permethrin in combination with fipronil and an insect growth regulator, such as s-methoprene and/or a juvenile hormone analog, such as pyriproxifen, are effective in producing a robust early knock down kill of fleas, while at the same time providing long term efficacy without increasing the volume of the solution administered. This is particularly surprising in view of the current view that more than 40% w/w of permethrin is necessary to achieve this quick kill without compromising long term efficacy in a spot-on formulation. Since permethrin can be used in the solution at a concentration of less than 40% w/w, or even less than about 20% w/w, the overall volume of the spot-on treatment, remains the same as currently marketed PetArmor Plus™ products and the like.

Furthermore, the present spot-on formulation has improved safety with respect to shipping, storage and handler safety while maintaining parasiticidal efficacy. In certain embodiments, the present formulations have been shown to have flash points from about 45° C. to about 55° C. and are therefore safer than the known compositions of the prior art, such as the FRONTLINE® products which have flash points of about 36° C. (97° F.). The term “flash point” as used herein denotes the minimum temperature (at least 40° C.) at which a spot-on formulation can form an ignitable mixture. The flash point can be determined by various methods known in the art. The flash point of the present spot-on formulations were determined by well-known Abel Cup method.

### Active Ingredient

As provided above, the formulations provided herein contain either fipronil (or a veterinary acceptable salt thereof) or a combination of fipronil (or a veterinary acceptable salt thereof) and S-methoprene (or a veterinary acceptable salt thereof).

The term “veterinary acceptable salt”, as used herein, refers to a salt prepared from veterinary acceptable non-toxic acids or bases including inorganic or organic acids and bases. Veterinary acceptable salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts. Suitable base salts include, but are not limited to, aluminum, calcium, lithium, magnesium, potassium, sodium, zinc, and diethanolamine salts.

Fipronil, or a veterinary acceptable salt thereof, in one embodiment, is present in the formulation at about 5% to about 15% w/w of the formulation. For example, in one embodiment, fipronil is present at about 5% to about 14% w/w, or about 6% to about 13% w/w, or about 7% to about 12% w/w, or about 8% to about 11% w/w, or about 9% to about 11% w/w of the formulation. In a further embodiment, fipronil is present at about 9.8% w/w of the formulation.

An insect growth regulator or a veterinary acceptable salt thereof, in one embodiment, is present in a co-formulation with fipronil at about 5% to about 20% w/w of the formulation. For example, in one embodiment, the insect growth regulator is present at about 5% to about 15% w/w, or about or about 6% to about 14% w/w, or about 7% to about 13% w/w, or about 8 to about 12% w/w. In a further embodiment, the insect growth regulator is present at about 7% or about 7.5% or about 8% or about 8.5% or about 9% or about 10% or about 11% w/w or about 12% w/w of the formulation. In a further embodiment, the insect growth regulator is present at about 8.8% w/w of the formulation. In another embodiment, the insect growth regulator is present at about 11.8% w/w of the formulation. In a further embodiment, the insect growth regulator is s-methoprene.

A juvenile hormone analogue or a veterinary acceptable salt thereof, in one embodiment, is present in a co-formulation with fipronil at about 0.5% to about 10% w/w of the formulation. For example, in one embodiment, the juvenile hormone analogue is present at about 1% to about 10% w/w, or about 2% to about 8% w/w, or about 3% to about 5% w/w or about 1% to about 3% w/w of the formulation of the formulation. In a further embodiment, the juvenile hormone analogue is present at about 0.5% or about 1% or about 1.5% or about 2% or about 2.5% or about 3% w/w or about 4% w/w or about 5% w/w or about 6% w/w or about 7% w/w or about 8% w/w or about 9% w/w or about 10% w/w of the formulation. In certain embodiments, the juvenile hormone analogue is pyriproxifen.

In another embodiment, the present spot-on formulations comprise a knock-down agent as an additional active ingredient. As used herein, the term “knock-down agent” refers to the chemical agents that function as neurotoxins to insects and produce a quick knockdown effect on insect pest populations. One family of knock-down agents is pyrethroids. One example of pyrethroids is permethrin or a veterinary acceptable salt thereof. The permethrin or a veterinary acceptable salt thereof is present in the formulation at a concentration to effectively produce a knock-down effect either alone or in combination with other active ingredient(s). In one embodiment, the permethrin or a veterinary acceptable salt thereof is present in an amount of about 40% or less w/w of the composition. In another embodiment, the permethrin or a veterinary acceptable salt thereof is present in an amount of about 35% or less w/w of the composition. In another embodiment, the permethrin or a veterinary acceptable salt thereof is present in an amount of about 30% or less w/w of the composition. In another embodiment, the permethrin or a veterinary acceptable salt thereof is present in an amount of about 25% or less w/w of the composition. In another embodiment, the permethrin or a veterinary acceptable salt thereof is present in an amount of about 20% or less w/w of the composition. In one embodiment, the present-spot on formulation comprises a potentiator of permethrin. One example of a potentiator of permethrin is piperonyl butoxide. In one embodiment, the piperonyl butoxide is present in an amount of about 0.1% w/w to about 20% w/w of the formulation.

In one embodiment, a formulation of the invention comprises fipronil, or a veterinary acceptable salt thereof at about 9.8% w/w of the formulation. In another embodiment, a formulation of the invention comprises fipronil, or a veterinary acceptable salt thereof at about 9.8% w/w of the formulation and S-methoprene, or a veterinary acceptable salt thereof at about 8.8% w/w of the formulation. In another embodiment, a formulation of the invention comprises fipronil, or a veterinary acceptable salt thereof at about 9.8% w/w of the formulation and S-methoprene, or a veterinary acceptable salt thereof at about 11.8% w/w of the formulation.

In one embodiment, a fipronil formulation is provided. The formulation comprises fipronil, at least one organic solvent, at least one antioxidant, and optionally, a C₁-C₆ alcohol co-solvent. In a further embodiment, the solvent is diethylene glycol monoethyl ether.

In another embodiment, a fipronil/S-methoprene co-formulation is provided. The formulation comprises fipronil, S-methoprene, at least one organic solvent, at least one antioxidant, and optionally, a C₁-C₆ alcohol co-solvent. In a further embodiment, the solvent is diethylene glycol monoethyl ether.

In one embodiment, a fipronil formulation is provided. The formulation comprises fipronil, at least one organic solvent, at least one antioxidant, and optionally, a C₁-C₆ alcohol co-solvent. In a further embodiment, the at least one antioxidant is BHA, BHT, and α-Tocopherol. In a further embodiment, the solvent is diethylene glycol monoethyl ether.

In one embodiment, a fipronil/S-methoprene co-formulation is provided. The formulation comprises fipronil, S-methoprene, at least one organic solvent, at least one antioxidant, and optionally, a C₁-C₆ alcohol co-solvent. In a further embodiment, the at least one antioxidant is BHA, BHT, and α-Tocopherol. In a further embodiment, the solvent is diethylene glycol monoethyl ether.

In another embodiment, a fipronil/S-methoprene/permethrin co-formulation is provided. The formulation comprises fipronil, S-methoprene, permethrin, at least one organic solvent, at least one antioxidant, and optionally, a C₁-C₆ alcohol co-solvent. In a further embodiment, the solvent is diethylene glycol monoethyl ether. In a further embodiment, the co-formulation also comprises piperonyl butoxide.

In another embodiment, a fipronil/pyriproxifen/permethrin co-formulation is provided. The formulation comprises fipronil, pyriproxifen, permethrin, at least one organic solvent, at least one antioxidant, and optionally, a C₁-C₆ alcohol co-solvent. In a further embodiment, the solvent is diethylene glycol monoethyl ether. In a further embodiment, the co-formulation also comprises piperonyl butoxide.

In one embodiment, a fipronil/S-methoprene/permethrin co-formulation is provided. The formulation comprises fipronil, S-methoprene, permethrin, at least one organic solvent, at least one antioxidant, and optionally, a C₁-C₆ alcohol co-solvent. In a further embodiment, the at least one antioxidant is BHA, BHT, and α-Tocopherol. In a further embodiment, the solvent is diethylene glycol monoethyl ether. In a further embodiment, the co-formulation also comprises piperonyl butoxide.

In one embodiment, a fipronil/pyriproxifen/permethrin co-formulation is provided. The formulation comprises fipronil, S-methoprene, pyriproxifen, at least one organic solvent, at least one antioxidant, and optionally, a C₁-C₆ alcohol co-solvent. In a further embodiment, the at least one antioxidant is BHA, BHT, and α-Tocopherol. In a further embodiment, the solvent is diethylene glycol monoethyl ether. In a further embodiment, the co-formulation also comprises piperonyl butoxide.

### Organic Solvent

The formulations of the invention contain an organic solvent. In one embodiment, the amount of the solvent ranges from about 40% to qs to 100% w/w of the formulation. By “qs to 100%”, it is meant adding the solvent to the formulation until a total of 100% of the formulation is achieved. In another embodiment, the amount of the solvent ranges from about 80% to qs to 100% or from about 85% to qs to 100% by weight of the formulation. The solvent is present, in one embodiment, at about 76% to about 90% w/w of the formulation, or about 76% to about 86% w/w of the formulation.

In one embodiment, the solvent is a glycol ether. By “glycol ether”, it is meant an ether compound derived from one or more hydroxyl groups of a glycol. Examples of suitable glycol ether solvent include, but are not limited to, dipropylene glycol n-butyl ether, ethylene glycol monoethyl ether, ethylene glycol monomethyl ether, monomethylacetamide, dipropylene glycol monomethyl ether, diethylene glycol monoethyl ether, dipropylene glycol n-butyl ether, and dipropylene glycol monomethyl ether. In one embodiment, the glycol ether is selected from diethylene glycol monoethyl ether, dipropylene glycol n-butyl ether, dipropylene glycol monomethyl ether, and any combination thereof.

In one specific embodiment, the glycol ether is diethylene glycol monoethyl ether (e.g., Transcutol P). Diethylene glycol monoethyl ether is also referred to herein as 2-(2-ethoxyethoxy) ethanol.

### Alcohol Co-Solvent

The present formulation contains up to about 10% alcohol co-solvent, i.e., from 0% to about 10% alcohol by weight of the formulation. In one embodiment, the present formulation contains up to about 5% alcohol co-solvent, i.e. from 0% to about 5% by weight of the formulation. That is, the present formulation may or may not contain an alcohol co-solvent. In some embodiments, the formulation contains an alcohol co-solvent, while in other embodiments, the formulation does not contain any alcohol co-solvent. Alcohol co-solvents can be present in fipronil formulations, as well as fipronil/S-methoprene co-formulations, and also can be present in formulations comprising one or more crystallization inhibitors.

In one embodiment, the alcohol co-solvent is present and is a C₁-C₆ alcohol. In one embodiment, the amount of the alcohol solvent ranges from about 1% to about 10% w/w of the formulation. In one embodiment, the amount of the alcohol solvent ranges from about 2% to about 5% w/w of the formulation. In one embodiment, the amount of the alcohol solvent ranges from about 3% to about 5% w/w of the formulation. In one embodiment, the amount of alcohol solvent is about 5% w/w of the formulation. Examples of the alcohol solvent include, but are not limited to, methanol, ethanol, propanol, isopropanol, butanol, isobutanol, t-butanol, and any combinations thereof. In one specific embodiment, the alcohol solvent is ethanol, isopropanol, or a combination thereof.

In one embodiment, an alcohol co-solvent is present at 5% w/w of the formulation. In a further embodiment, the co-solvent is methanol, ethanol, propanol or isopropanol. In even a further embodiment, the co-solvent is ethanol or isopropanol. In one embodiment, a fipronil formulation is provided comprising fipronil at about 9.8% w/w of the formulation and ethanol at about 5% w/w of the formulation. In one embodiment, a fipronil/S-methoprene co-formulation is provided comprising fipronil at about 9.8% w/w of the formulation; S-methoprene at about 8.8% w/w of the formulation; and ethanol at about 5% w/w of the formulation.

### Antioxidant

In one embodiment, the present formulation contains at least one antioxidant. In one embodiment, the amount of antioxidant in the present formulation ranges from about 0.005 to about 1% by weight of the formulation. In another embodiment, the amount of antioxidant in the present formulation ranges from about 0.005% to about 0.05% by weight of the formulation. In another embodiment, the amount of antioxidant in the present formulation ranges from about 0.01% to about 0.04% by weight of the formulation.

In one embodiment, the at least one antioxidant in the present formulation is about 0.03% by weight of the formulation. In one embodiment, the at least one antioxidant in the present formulation is about 0.04% by weight of the formulation. In a further embodiment, the present formulation contains two or three antioxidants.

Examples of the antioxidant include, but are not limited to, butylated hydroxylanisole (BHA), butylated hydroxyltoluene (BHT), alpha-tocopherol (α-tocopherol), ascorbic acid, ascobyl palmitate, tumeric acid, malic acid, citric acid, sodium ascorbate, sodium metabisulfate, n-propyl gallate, monothioglycerol and combinations thereof. In one embodiment, the antioxidant is butylated hydroxylanisole, butylated hydroxyltoluene, alpha-tocopherol, and any combinations thereof. The α-tocopherol may be in various stereoisomeric forms due the chiral centers in the molecule. In the present formulation, the α-tocopherol can be any of the enantiomers; combinations of enantiomers, diastereomers, or stereoisomers; or a racemic mixture.

In one embodiment, the present formulation contains both BHA and BHT. In one embodiment, the present formulation contains BHA, BHT, and alpha-tocopherol. In another embodiment, BHA and BHT are present at about 0.03% w/w of the formulation. In a further embodiment, BHA is present at about 0.02% w/w and BHT is present at about 0.01% w/w of the formulation. In even a further embodiment, BHA is present at about 0.02% w/w, BHT is present at about 0.01% w/w, and alpha-tocopherol is present at about 0.01% w/w of the formulation.

The present spot-on formulations may also comprise one or more additional other veterinary acceptable excipients, such as those excipient which can sooth the skin, e.g., chamomile or chamomile extracts, aloe, and etc.t.

### Crystallization Inhibitor

As provided above, the present formulation does not contain any surfactant or crystallization inhibitor, although other embodiments may contain crystallization inhibitor(s). A “crystallization inhibitor” refers to an agent in a formulation which prevents crystallization of the active ingredient from the formulation. For example, a crystallization inhibitor can prevent crystallization of a drug in a formulation in the container or the hair or skin of the animal.

The crystallization inhibitor may be a nonionic, cationic, anionic, or amphoteric surfactant or any combination thereof.

Nonionic surfactants include, but are not limited to, polyoxyethylenated sorbitan esters, such as polysorbate 80; polyoxyethylenated alkyl ethers; polyethylene glycol stearate; polyoxyethylenated derivatives of castor oil (i.e., polyoxyethylenated castor oil); polyglycerol esters; polyoxyethylenated fatty alcohols; polyoxyethylenated fatty acids; copolymers of ethylene oxide; and propylene oxide.

Cationic surfactants include, but are not limited to, water-soluble quaternary ammonium salts, such as cetyltrimethylammonium bromide, and octadecylamine hydrochloride.

Anionic surfactants include, but are not limited to, alkaline stearates, such as sodium, potassium or ammonium stearate; calcium stearate; triethanolamine stearate; sodium abietate; alkyl sulphates, in particular sodium lauryl sulphate and sodium cetyl sulphate; sodium dodecylbenzenesulphonate, sodium dioctylsulphosuccinate; fatty acids, such as those derived from coconut oil.

Amphoteric surfactants include, but are not limited to, the substituted lauryl compounds of betaine.

In addition, the crystallization inhibitor may also be selected from the group consisting of polyvinylpyrrolidone, polyvinyl alcohols, copolymers of vinyl acetate and vinylpyrrolidone, polyethylene glycols (PEG), benzyl alcohol, mannitol, glycerol, sorbitol, polyoxyethylenated sorbitan esters; lecithin, sodium carboxymethylcellulose; and acrylic derivatives such as methacrylates.

In one embodiment, the present formulation also excludes polyoxyethylene castor oil as the crystallization inhibitor. Polyoxyethylene castor oils are widely used in oral, topical, and parenteral pharmaceutical and veterinary formulations as emulsifying and solubilizing agents for the aqueous preparations containing lipophilic ingredients. Those compounds are complex mixtures of various hydrophobic and hydrophilic components. Examples of those compounds include, but are not limited to, polyoxyethylene 5 castor oil (Acconon CA-5), polyoxyethylene 9 castor oil (Acconon CA-9), polyoxyethylene 15 castor oil (Acconon CA-15), polyoxyethylene 35 castor oil (Cremophor EL, Cremophor ELP, Etocas 35), polyoxyethylene 40 castor oil, polyoxyl 40 hydrogenated castor oil (Cremophor RH 40, Emulgin HRE 40), polyoxyl 40 hydrogenated castor oil (Emulgin HRE 60). The surfactant, in one embodiment, is a polyoxyethylene 35 castor oil or a polyoxyethylene 40 castor oil.

In one embodiment, the present formulation excludes a polyethylene glycol as the crystallization inhibitor. The term “polyethylene glycol”, as used herein, includes both a polyethylene glycol with a particular molecular weight and any combinations of polyethylene glycols having different molecular weights.

In another embodiment, the present formulation excludes both a polyoxyethylenated castor oil and a polyethylene glycol (e.g., PEG 400).

### Specific Embodiments and Experiments

In one specific embodiment, the present spot-on formulation comprises about 9.8% w/w of fipronil; about 5% w/w of ethanol; about 0.02% w/w of BHA; about 0.01% w/w of BHT; about 0.01% w/w of α-Tocopherol; and balance diethylene glycol monoethyl ether; and does not contain any surfactant and crystallization inhibitor. In another embodiment of the formulation, it further comprises about 8.8% w/w of S-methoprene.

Select Specific formulations of the invention are provided in Table 1, below:

TABLE 1
Specific formulations of the invention.

| | Formulation 1 | | Formulation 2 | | Formulation 3 | |
|---|---|---|---|---|---|---|
| Active(s) | Fipronil | 9.8% | Fipronil | 9.8% | Fipronil | 9.8% |
| | S-Methoprene | 8.8% | | | S-Methoprene | 8.8% |
| C₁-C₆ Alcohol | Ethanol | 5% | Ethanol | 5% | — | — |
| Antioxidant(s) | BHA | 0.02% | BHA | 0.02% | BHA | 0.02% |
| | BHT | 0.01% | BHT | 0.01% | BHT | 0.01% |
| | α-Tocopherol | 0.01% | α-Tocopherol | 0.01% | | |
| solvent | diethylene glycol | 76.36% | diethylene glycol | 85.16% | diethylene glycol | 81.37% |
| | monoethyl ether¹ | | monoethyl ether¹ | | monoethyl ether¹ | |
| | Formulation 4 | | Formulation 5 | | Formulation 6 | |
| Active(s) | Fipronil | 9.8% | Fipronil | 9.8% | Fipronil | 9.8% |
| | S-Methoprene | 8.8% | | | | |
| C₁-C₆ Alcohol | Ethanol | 5% | Ethanol | 5% | Isopropanol | 5% |
| Antioxidant(s) | BHA | 0.02% | BHA | 0.02% | BHA | 0.02% |
| | BHT | 0.01% | BHT | 0.01% | BHT | 0.01% |
| solvent | diethylene glycol | 76.37% | diethylene glycol | 85.17% | diethylene glycol | 85.17% |
| | monoethyl ether¹ | | monoethyl ether¹ | | monoethyl ether¹ | |

¹e.g., Transcutol P

The specific formulations of the present invention, such as the ones listed in the above Table 1, have been tested and shown to retain efficacy. That is, the present spot-on formulation have demonstrated its efficacy, its speed of action, and the pleasant appearance of the animal's hair after application and drying. Once deposited, the composition dries and diffuses over the mammal's body without crystallizing or modifying the appearance or feel of the fur.

In the Comparative Efficacy Studies I, II, III, IV, V, and VI reported below, various batches of FD101 contain about 9.8% fipronil; about 5% ethanol; antioxidants; diethylene glycol monoethyl ether (balance of the formulation); and without surfactant and crystallization inhibitor; various batches of FD101 PLUS contain about 9.8% fipronil; about 8.8% S-methoprene; about 5% ethanol; antioxidants; diethylene glycol monoethyl ether (balance of the formulation); and without surfactant and crystallization inhibitor; various Frontline® Top Spot formulations contain about 9.7% fipronil; about 10% ethanol; crystallization inhibitor(s); antioxidant(s); and other solvents (balance of the formulation); and various batches of Frontline® PLUS formulations contain about 9.8% fipronil; about 8.8% S-methoprene; about 10% ethanol; crystallization inhibitor(s); antioxidant(s); other solvents (balance of the formulation).

In one specific embodiment, the present spot-on formulation comprises about 5-12% w/w of fipronil; about 7-12% s-methoprene; less than about 40% permethrin; about 5% w/w of ethanol; about 0.02% w/w of BHA; about 0.01% w/w of BHT; about 0.01% w/w of α-Tocopherol; and balance diethylene glycol monoethyl ether; and does not contain any surfactant and crystallization inhibitor. In a further embodiment, the formulation also comprises piperonyl butoxide.

In one specific embodiment, the present spot-on formulation comprises about 5-12% w/w of fipronil; about 7-12% s-methoprene; less than about 20% permethrin; about 5% w/w of ethanol; about 0.02% w/w of BHA; about 0.01% w/w of BHT; about 0.01% w/w of α-Tocopherol; and balance diethylene glycol monoethyl ether; and does not contain any surfactant and crystallization inhibitor. In a further embodiment, the formulation also comprises piperonyl butoxide.

In one specific embodiment, the present spot-on formulation comprises about 5-12% w/w of fipronil; about 1-10% pyriproxifen; less than about 40% permethrin; about 5% w/w of ethanol; about 0.02% w/w of BHA; about 0.01% w/w of BHT; about 0.01% w/w of α-Tocopherol; and balance diethylene glycol monoethyl ether; and does not contain any surfactant and crystallization inhibitor. In a further embodiment, the formulation also comprises piperonyl butoxide.

In one specific embodiment, the present spot-on formulation comprises about 5-12% w/w of fipronil; about 1-10% pyriproxifen; less than about 20% permethrin; about 5% w/w of ethanol; about 0.02% w/w of BHA; about 0.01% w/w of BHT; about 0.01% w/w of α-Tocopherol; and balance diethylene glycol monoethyl ether; and does not contain any surfactant and crystallization inhibitor. In a further embodiment, the formulation also comprises piperonyl butoxide.

The specific formulations of the present invention, such as those listed above containing permethrin in addition to fipronil, and s-methoprene and/or pyriproxifen will demonstrate a quick knock down as well as prolonged efficacy in the killing of fleas. That is, the spot-on formulations of the current invention will demonstrate efficacy, speed of action, and the pleasant appearance of the animal's hair after application and drying. Once deposited, the composition will dry and diffuses over the mammal's body without crystallizing or modifying the appearance or feel of the fur.

In certain embodiments, the spot-on formulations of the present invention will display about 90% or greater flea death within about 3 hours of application and about 30 days of protection against flea re-infestation. In a particular embodiment, the spot on formulation of the present invention, while containing an additional active agent, permethrin, as compared to the currently marketed Pet Armor Plus™, will still be applied in the same volume of solution as the Pet Armor Plus™ product.

### Comparative Efficacy Study—I

### Objective

To determine and compare the adulticidal efficacy against ticks (Amblyomma americanum and Ixodes scapularis) of a fipronil spot-on formulation of the present invention to that of Frontline® Top Spot® for dogs, when administered topically to dogs.

### Study Design and Groups

This study was a parallel group design, randomized, unicentre, blinded controlled efficacy study. In order to control bias, the animals were treated by an individual not involved in performing the post-treatment assessments and observations. Study groups were coded to blind the performing post-treatment observations and assessments.

The study was conducted on three groups of eight dogs each.
Group 1: Untreated control
Group 2: Dogs were treated with the IVP (FD101) at a dosage of 0.067 ml/kg b.w.
Group 3: Dogs were treated with the CVP (Frontline® Top Spot® for dogs) at a dosage of 0.067 ml/kg b.w.

### Study Layout

| | | Ranking and | Administration | |
|---|---|---|---|---|
| Acclima- | Tick | Allocation to | of | Tick |
| tization | Infestations | Groups | IVP/CVP | Count* |
| Days −7 | Days −6 | Day −3 | Day 0 | Days −4, |
| to −1 | (A. americanum | | | +2, +9, |
| | only) −1, +7, | | | +16, +23 |
| | +14, +21 and +30 | | | and +32 |

*Tick counts were conducted 48 (± 4) hours post-treatment or infestation

### Randomization

The study followed a randomized block design. On Day −3 the 24 dogs included were ranked, within gender, in descending order of individual pre-treatment tick counts. Lottery was used to break ties. Within each gender, animals were then formed into replicates of three dogs each. Within each block, dogs were randomly allocated to Groups 1, 2 or 3. The groups were color coded to blind the post-treatment assessments.

### Treatments

Treatments were as set out below:

| Study | Sample | | Active | | Appli- | |
|---|---|---|---|---|---|---|
| Group | Size | IVP/CVP | Ingredient | Dosages | cation | Day |
| 2 | 8 | FD101 | Fipronil | 0.067 | Topical | 0 |
| | | | | ml/kg b.w. | spot-on | |
| 3 | 8 | Frontline ® | Fipronil | 0.067 | Topical | 0 |
| | | Top Spot ® | | ml/kg b.w. | spot-on | |
| | | for dogs | | | | |

### Study Procedures

### Tick Infestations

Laboratory-bred strains of Amblyomma americanum and Ixodes scapularis were used in the artificial infestations. Immature ticks were fed on rabbits. Adult ticks, which were used in the artificial infestations, were unfed, at least 3 weeks old and had a balanced sex ratio (−50% female:˜50% male). On Day −6, each dog was artificially infested with 50 Amblyomma americanum ticks. Thereafter, each dog was artificially infested with 50 ticks of each species on Days −1, +7, +14, +21 and +30. The time of infestation was recorded for all animals.

### Tick Counts

The time at which each animal was treated or at which it was infested with ticks was recorded. This was done to ensure that counting and removal of ticks was as close as possible to the specified target times (48±4 hours post infestation or treatment). The time of tick counting and removal was recorded. Ticks were found by direct observation following parting of the hair coat and palpation. Areas examined, not necessarily in this order, were the following:

Outside hind legs, including feet
Tail and anal areas
Lateral area, not including shoulders
Abdominal area, from chest to inside hind legs
Forelegs and shoulders, including feet
All neck and head areas
Dorsal strip from shoulder blades to base of tail

Ticks removed were recorded on the appropriate data capture form according to the parameters given below:

| Category | General Findings | Attachment Status |
|---|---|---|
| 1 | Live | Free |
| 2 | Live | Attached; unengorged* |
| 3 | Live | Attached; engorged** |
| 4 | Killed | Free |
| 5 | Killed | Attached; unengorged* |
| 6 | Killed | Attached; engorged** |

*no filling of the alloscutum evident
**obvious or conspicuous filling of the alloscutum evident

All dogs were combed following the 48-hour tick counts and removal to ensure that all ticks were counted and removed.

### Statistical Methods

The efficacy against ticks was calculated for the treatment groups at each assessment day according to the formulas given below. Due to the fact that small and even zero tick counts were recorded it was expected that the tick counts would not follow a normal distribution. It was therefore decided that the primary efficacy calculations would be based on geometric means rather than arithmetic means. The calculations were based on the geometric means of the tick (count+1) data. One (1) was subsequently subtracted from the result to obtain a meaningful value for the geometric mean of each group. Efficacy calculations based on arithmetic means are also reported.

Efficacy against ticks was calculated according to the following formula:

Efficacy(%)=100×(m_{c}−mₜ)/m_{c}, where

m_{c}=Geometric mean number of live ticks (categories 1-3) on dogs in the untreated control group (Group 1) at a specific time point.
mₜ=Geometric mean number of live and dead ticks (categories 1-3 & 6) on dogs in the treatment group (Groups 2 and 3) at a specific time point.

Descriptive statistics (mean, minimum, maximum, standard deviation, CV %, geometric mean and median) on tick counts for the various assessment days were calculated.

### Results

No adverse reaction was observed to any of the treatments at any time during the study.

### Tick Counts

Ixodes scapularis

Arithmetic and geometric mean Ixodes scapularis tick counts on the various assessment days for the three study groups are summarized below. The arithmetic mean tick counts recorded for the untreated control group ranged from 14.8 to 18.0 indicating vigorous tick challenges on all assessment days.

| | | | | | Group 3-CVP | |
|---|---|---|---|---|---|---|
| | Group 1-Untreated | | Group 2-IVP | | (Frontline ® Top | |
| | Control | | (FD101) | | Spot ® for dogs) | |
| | Arith- | Geo- | Arith- | Geo- | Arith- | Geo- |
| | metic | metric | metic | metric | metic | metric |
| Day | Mean | Mean | Mean | Mean | Mean | Mean |
| +2 | 15.0 | 14.5 | 4.8 | 2.8 ^{A} | 10.3 | 7.5 |
| +9 | 17.0 | 16.4 | 0.4 | 0.3 ^{A} | 0.0 | 0.0 ^{A} |
| +16 | 14.8 | 14.6 | 1.1 | 0.8 ^{A} | 0.8 | 0.4 ^{A} |
| +23 | 17.0 | 16.9 | 1.0 | 0.7 ^{A} | 0.5 | 0.4 ^{A} |
| +32 | 18.0 | 17.9 | 2.0 | 1.3 ^{A} | 1.1 | 1.0 ^{A} |

^{A} Significantly different from control (p < 0.01)

There was no significant (p>0.10) difference between FD 101 and Frontline® Top Spot® for dogs on any examination day.

Amblyomma americanum

Arithmetic and geometric mean Amblyomma americanum tick counts on the various assessment days for the three study groups are summarized below. The arithmetic mean tick count recorded for the untreated control group ranged from 13.9 to 20.8 indicating vigorous tick challenges on all assessment days. The geometric mean tick counts recorded for both treatment groups differed significantly (p<0.05) from that of the untreated control group on all assessment days.

| | | | | | Group 3-CVP | |
|---|---|---|---|---|---|---|
| | Group 1-Untreated | | Group 2-IVP | | (Frontline ® Top | |
| | Control | | (FD101) | | Spot ® for dogs) | |
| | Arith- | Geo- | Arith- | Geo- | Arith- | Geo- |
| | metic | metric | metic | metric | metic | metric |
| Day | Mean | Mean | Mean | Mean | Mean | Mean |
| +2 | 13.9 | 12.9 | 1.3 | 0.8 ^{A} | 3.0 | 1.3 ^{A} |
| +9 | 15.8 | 15.4 | 0.1 | 0.1 ^{A} | 0.0 | 0.0 ^{A} |
| +16 | 20.0 | 19.9 | 0.0 | 0.0 ^{A} | 0.0 | 0.0 ^{A} |
| +23 | 20.8 | 20.6 | 0.3 | 0.1 ^{A} | 0.0 | 0.0 ^{A} |
| +32 | 20.4 | 20.2 | 0.6 | 0.4 ^{A} | 0.1 | 0.1 ^{A} |

^{A} Significantly different from control (p < 0.01)

There was no significant (p>0.10) difference between FD 101 and Frontline® Top Spot® for dogs on any examination day.

### Efficacy Data

Ixodes scapularis

Efficacy values (%) based on arithmetic and geometric means for the groups treated with the investigational and control veterinary products against Ixodes scapularis are summarized below.

| | EFFICACIES (%) Ixodes scapularis | | | |
|---|---|---|---|---|
| | | | GROUP 3 - CVP | |
| | GROUP 2 - | | (Frontline ® Top | |
| | IVP (FD 101) | | Spot ® for dogs) | |
| | Arithmetic | | Arithmetic | |
| DAY | Mean | Geometric Mean | Mean | Geometric Mean |
| +2 | 68.0 | 80.6 | 31.3 | 48.1 |
| +9 | 97.6 | 98.5 | 100.0 | 100.0 |
| +16 | 92.6 | 94.4 | 94.6 | 97.2 |
| +23 | 94.1 | 95.8 | 97.1 | 97.8 |
| +32 | 88.9 | 92.6 | 93.9 | 94.7 |

Dogs treated with FD 101 had significantly (p<0.01) fewer ticks than the controls at each post-treatment examination. Dogs treated with Frontline® Top Spot® for dogs had significantly (p<0.01) fewer ticks than the controls from Day 9 through the end of the study; there was no significant (p>0.10) difference between the two treated groups at any examination.

Amblyomma americanum

Efficacy values (%) based on arithmetic and geometric means for the groups treated with the investigational and control veterinary products against Amblyomma americanum are summarized below.

| | EFFICACIES (%) Amblyomma americanum | | | |
|---|---|---|---|---|
| | | | GROUP 3 - CVP | |
| | GROUP 2 - IVP | | (Frontline ® Top | |
| | (FD 101) | | Spot ® for dogs) | |
| | Arithmetic | | Arithmetic | |
| DAY | Mean | Geometric Mean | Mean | Geometric Mean |
| +2 | 90.6 | 93.8 | 78.4 | 90.1 |
| +9 | 99.4 | 99.4 | 100.0 | 100.0 |
| +16 | 100.0 | 100.0 | 100.0 | 100.0 |
| +23 | 98.6 | 99.3 | 100.0 | 100.0 |
| +32 | 97.1 | 97.9 | 99.5 | 99.6 |

The IVP (FD 101) and CVP (Frontline® Top Spot® for dogs) were similarly effective against challenges with Amblyomma americanum ticks throughout the 32-day post-treatment observation period.

### Conclusion

The IVP (FD 101) and CVP (Frontline® Top Spot® for dogs) administered at a dosage of 0.067 ml/kg b.w. to dogs had comparable immediate and persistent efficacies against challenges with Amblyomma americanum and Ixodes scapularis ticks from Day 7 through Day 30 of the trial.

The mean count of Ixodes scapularis in the CVP (Frontline® Top Spot® for dogs) group was not significantly different from the control value at Day +2 following treatment.

### Comparative Efficacy Study—II

### Objective

To determine and compare the adulticidal efficacy against ticks (Amblyomma americanum and Ixodes scapularis) of a fipronil and s-methoprene spot-on formulation of the present invention to that of Frontline® Plus for dogs, when administered topically to dogs.

### Study Design and Groups

This study was a parallel group design, randomized, unicentre, blinded controlled efficacy study. In order to control bias, the animals were treated by an individual not involved in performing the post-treatment assessments and observations. Study groups were coded to blind the post-treatment observations and assessments.

The study was conducted on three groups of eight dogs each.

Group 1: Untreated control
Group 2: Dogs were treated with the IVP (FD 101 PLUS) at a dosage of 0.067 ml/kg b.w.
Group 3: Dogs were treated with the CVP (Frontline® Plus for dogs) at a dosage of 0.067 ml/kg b.w.

### Study Layout

| | | Ranking and | Administration | |
|---|---|---|---|---|
| Acclima- | Tick | Allocation to | of | Tick |
| tization | Infestations | Groups | IVP/CVP | Count* |
| Days −7 | Days −6 | Day −3 | Day 0 | Days −4, |
| to −1 | (A. americanum | | | +2, +9, |
| | only) −1, | | | +16, +23 |
| | +7, +14, +21 | | | and +32 |
| | and +30 | | | |

*Tick counts were conducted 48 (±4) hours post-treatment or infestation

### Randomization

The study followed a randomized block design. On Day −3 the 24 dogs included were ranked, within gender, in descending order of individual pre-treatment tick counts. Lottery was used to break ties. Within each gender, animals were then formed into replicates of three dogs each. Within each block, dogs were randomly allocated to Groups 1, 2 or 3. The groups were color coded to blind the post-treatment assessments.

### Treatments

Treatments were as set out below:

| Study | Sample | | Active | | Appli- | |
|---|---|---|---|---|---|---|
| Group | Size | IVP/CVP | Ingredient | Dosages | cation | Day |
| 2 | 8 | FD 101 | Fipronil | 0.067 | Topical | 0 |
| | | PLUS | and s- | ml/kg b.w. | spot on | |
| | | | meth- | | | |
| | | | oprene | | | |
| 3 | 8 | Frontline ® | Fipronil | 0.067 | Topical | 0 |
| | | Plus | and s- | ml/kg b.w. | spot-on | |
| | | for dogs | meth- | | | |
| | | | oprene | | | |

### Study Procedures

### Tick Infestations

Laboratory-bred strains of Amblyomma americanum and Ixodes scapularis were used in the artificial infestations. Immature ticks were fed on rabbits. Adult ticks, which were used in the artificial infestations, were unfed, at least 3 weeks old and had a balanced sex ratio (−50% female:˜50% male). On Day −6, each dog was artificially infested with 50 Amblyomma americanum ticks. Thereafter, each dog was artificially infested with 50 ticks of each species on Days −1, +7, +14, +21 and +30. The time of infestation was recorded for all animals.

### Tick Counts

The time at which each animal was treated or at which it was infested with ticks was recorded. This was done to ensure that counting and removal of ticks was as close as possible to the specified target times (48±4 hours post infestation or treatment). The time of tick counting and removal was recorded. Ticks were found by direct observation following parting of the hair coat and palpation. Areas examined, not necessarily in this order, were the following:

Outside hind legs, including feet
Tail and anal areas
Lateral area, not including shoulders
Abdominal area, from chest to inside hind legs
Forelegs and shoulders, including feet
All neck and head areas
Dorsal strip from shoulder blades to base of tail

Ticks removed were recorded on the appropriate data capture form according to the parameters given below:

| Category | General Findings | Attachment Status |
|---|---|---|
| 1 | Live | Free |
| 2 | Live | Attached; unengorged* |
| 3 | Live | Attached; engorged** |
| 4 | Killed | Free |
| 5 | Killed | Attached; unengorged* |
| 6 | Killed | Attached; engorged** |

*no filling of the alloscutum evident
**obvious or conspicuous filling of the alloscutum evident

All dogs were combed following the 48-hour tick counts and removal to ensure that all ticks were counted and removed.

### Statistical Methods

The efficacy against ticks was calculated for the treatment groups at each assessment day according to the formulas given below. Due to the fact that small and even zero tick counts were recorded it was expected that the tick counts would not follow a normal distribution. It was therefore decided that the primary efficacy calculations would be based on geometric means rather than arithmetic means. The calculations were based on the geometric means of the tick (count+1) data. One (1) was subsequently subtracted from the result to obtain a meaningful value for the geometric mean of each group. Efficacy calculations based on arithmetic means are also reported.

Efficacy against ticks was calculated according to the following formula:

Efficacy(%)=100×(m_{c}−mₜ)/m_{c}, where

m_{c}=Geometric mean number of live ticks (categories 1-3) on dogs in the untreated control group (Group 1) at a specific time point.
mₜ=Geometric mean number of live and dead ticks (categories 1-3 & 6) on dogs in the treatment group (Groups 2 and 3) at a specific time point.

Descriptive statistics (mean, minimum, maximum, standard deviation, CV %, geometric mean and median) on tick counts for the various assessment days were calculated.

### Results

No adverse reaction was observed to any of the treatments at any time during the study.

### Tick Counts

Ixodes scapularis

Arithmetic and geometric mean Ixodes scapularis tick counts on the various assessment days for the three study groups are summarized below. The arithmetic mean tick counts recorded for the untreated control group ranged from 13.3 to 18.8 indicating vigorous tick challenges on all assessment days.

| | | | | | Group 3-CVP | |
|---|---|---|---|---|---|---|
| | Group 1-Untreated | | Group 2-IVP | | (Frontline ® | |
| | Control | | (FD 101 PLUS) | | Plus for dogs) | |
| | Arith- | Geo- | Arith- | Geo- | Arith- | Geo- |
| | metic | metric | metic | metric | metic | metric |
| Day | Mean | Mean | Mean | Mean | Mean | Mean |
| +2 | 18.8 | 18.2 | 14.6 | 12.2 | 14.3 | 13.0 |
| +9 | 13.3 | 13.0 | 0.8 | 0.5^{B} | 0.8 | 0.5^{B} |
| +16 | 16.0 | 15.7 | 0.6 | 0.4^{B} | 0.4 | 0.3^{B} |
| +23 | 17.5 | 17.4 | 1.9 | 1.3^{B} | 2.3 | 1.5^{B} |
| +32 | 18.5 | 18.2 | 2.8 | 1.8^{B} | 3.0 | 1.8^{B} |

^{B}Significantly different from control (p < 0.01)

There was no significant (p>0.10) difference between FD 101 PLUS and Frontline® Plus for dogs on any examination day.

Amblyomma americanum

Arithmetic and geometric mean Amblyomma americanum tick counts on the various assessment days for the three study groups are summarized below. The arithmetic mean tick count recorded for the untreated control group ranged from 14.0 to 19.8 indicating vigorous tick challenges on all assessment days. The geometric mean tick counts recorded for both treatment groups differed significantly (p<0.01) from that of the untreated control group on all assessment days.

| | | | | | Group 3-CVP | |
|---|---|---|---|---|---|---|
| | Group 1-Untreated | | Group 2-IVP | | (Frontline ® | |
| | Control | | (FD 101 PLUS) | | Plus for dogs) | |
| | Arith- | Geo- | Arith- | Geo- | Arith- | Geo- |
| | metic | metric | metic | metric | metic | metric |
| Day | Mean | Mean | Mean | Mean | Mean | Mean |
| +2 | 14.0 | 13.8 | 6.0 | 5.7^{B} | 7.4 | 5.9^{A} |
| +9 | 14.5 | 14.2 | 0.0 | 0.0^{B} | 0.0 | 0.0^{B} |
| +16 | 19.8 | 19.6 | 0.0 | 0.0^{B} | 0.4 | 0.2^{B} |
| +23 | 19.1 | 19.0 | 0.3 | 0.2^{B} | 0.0 | 0.0^{B} |
| +32 | 19.8 | 19.4 | 0.6 | 0.3^{B} | 1.6 | 0.6^{B} |

^{A}Significantly different from control (p < 0.05
^{B}Significantly different from control (p < 0.01)

There was no significant (p>0.10) difference between FD 101 PLUS and Frontline® Plus for dogs on any examination day for either species of tick.

### Efficacy Data

Ixodes scapularis

Efficacy values (%) based on arithmetic and geometric means for the groups treated with the investigational and control veterinary products against Ixodes scapularis are summarized below.

| | EFFICACIES (%) Ixodes scapularis | | | |
|---|---|---|---|---|
| | GROUP 2 - IVP | | GROUP 3 - CVP | |
| | (FD 101 PLUS) | | (Frontline ® Plus for dogs) | |
| | Arithmetic | | Arithmetic | |
| DAY | Mean | Geometric Mean | Mean | Geometric Mean |
| +2 | 22.3 | 33.0 | 23.9 | 28.7 |
| +9 | 94.0 | 96.2 | 94.0 | 96.2 |
| +16 | 96.3 | 97.2 | 97.5 | 98.1 |
| +23 | 89.1 | 92.6 | 86.9 | 91.3 |
| +32 | 84.9 | 89.9 | 83.8 | 89.9 |

Dogs treated with FD 101 PLUS and dogs treated with Frontline® Plus for dogs had significantly (p<0.01) fewer ticks than the controls from Day 9 through the end of the study. There was no significant (p>0.10) difference between the two treated groups at any examination.

Amblyomma americanum

Efficacy values (%) based on arithmetic and geometric means for the groups treated with the investigational and control veterinary products against Amblyomma americanum are summarized below.

| | EFFICACIES (%) Amblyomma americanum | | | |
|---|---|---|---|---|
| | GROUP 2 - IVP | | GROUP 3 - CVP | |
| | (FD 101 PLUS) | | (Frontline ® Plus for dogs) | |
| | Arithmetic | | Arithmetic | |
| DAY | Mean | Geometric Mean | Mean | Geometric Mean |
| +2 | 57.1 | 58.4 | 47.1 | 56.8 |
| +9 | 100.0 | 100.0 | 100.0 | 100.0 |
| +16 | 100.0 | 100.0 | 98.0 | 99.0 |
| +23 | 98.4 | 99.0 | 100.0 | 100.0 |
| +32 | 97.0 | 98.3 | 91.9 | 97.1 |

The IVP (FD 101 PLUS) and CVP (Frontline® Plus for dogs) were similarly effective against challenges with Amblyomma americanum ticks throughout the 32-day post-treatment observation period.

### Conclusion

The IVP (FD 101 PLUS) and CVP (Frontline® Plus for dogs) administered at a dosage of 0.067 ml/kg b.w. to dogs had comparable immediate and persistent efficacies against challenges with Amblyomma americanum and Ixodes scapularis ticks from Day 7 through Day 30 of the trial.

CVP (Frontline® Plus for dogs) was not significantly different (p>0.01) from the control value at Day +2 following treatment for either species of tick.

### Comparative Efficacy Study—III

### Objectives

To determine and compare the adulticidal efficacy against fleas (Ctenocephalides felis) of a fipronil spot-on formulation of the present invention (PetArmor) to that of Frontline Top Spot for dogs, when administered to dogs.

### Study Design and Groups

This study was a parallel group design, randomised, unicentre, blinded controlled efficacy study. The study was conducted on three groups of eight dogs each.

Group 1: Untreated control
Group 2: Dogs treated with the IVP (PetArmor) at a dosage of 0.067 ml/kg b.w.
Group 3: Dogs treated with the CVP (Frontline Top Spot) at a dosage of 0.067 ml/kg b.w.

### Randomisation

The study followed a randomised block design.

### Treatments

In this study the IVP and CVP were applied once at the beginning of the study (Day 0).

Treatments followed the dose level as set out below:

| Study | Sample | | Active | | Appli- | |
|---|---|---|---|---|---|---|
| group | size | IVP/CVP | ingredient | Dosages | cation | Day |
| 2 | 8 | PetArmor | Fipronil | 0.067 | Topical | 0 |
| | | | | ml/kg b.w. | spot-on | |
| 3 | 8 | Frontline Top | Fipronil | 0.067 | Topical | 0 |
| | | Spot for Dogs | | ml/kg b.w. | spot-on | |

### Study Procedures

### Flea Infestations

A laboratory bred strain (PLRS US strain) of Ctenocephalides felis (routinely fed on cats) was used for all infestations. Fleas were unfed and of mixed sex. Each dog was infested with 100 fleas on Days −6, −1, +7, +14, +21 and +30. The fleas were not placed on or near the site of IVP/CVP application after treatment. The time of infestation was recorded for all animals.

### Flea Counts

Body flea counts were conducted as close as possible to the specified target times (48±2 hr post-treatment or infestation) on Days −4, +2, +9, +16, +23 and +32. The time of flea counting was recorded. During combing a fine-toothed flea comb was used to recover fleas present in the animal's fur. The method of combing was by several strokes of the comb on each area of the animal, each time moving in the same direction following the pattern of the hair coat. Movement, from one part of the animal's fur to the next was via strokes overlapping each other, so that no area of fur was missed. Areas to be examined, not necessarily in this order, were:

Outside hind legs, including feet
Tail and anal areas
Lateral area, not including shoulders
Abdominal area, from chest to inside hind legs
Forelegs and shoulders, including feet
All neck and head areas
Dorsal strip from shoulder blades to base of tail

After completion of the combing procedure for all body areas, the whole procedure was repeated once more so that all areas were combed a minimum of two times. When necessary, the combing procedure was continued for a third time or more until no live fleas were found.

### Statistical Methods

### Adulticidal Efficacy

The efficacy against fleas was calculated for the treatment groups at each assessment day according to the formulas given below. Due to the fact that small and even zero flea counts were recorded it was expected that the flea counts would not follow a normal distribution and so the primary efficacy calculations were based on geometric means rather than arithmetic means. The calculations were based on the geometric means of the flea (count+1) data and one (1) was subsequently subtracted from the result to obtain a meaningful value for the geometric mean of each group. Efficacy calculations based on arithmetic means were also included as part of the statistics package.

Efficacy against fleas were calculated according to the following formula:

Efficacy(%)=100×(m_{c}−mₜ)/m_{c}, where

m_{c}=geometric/arithmetic mean of live fleas on the negative control group (Group 1)
mₜ=geometric/arithmetic mean of live fleas on the IVP/CVP treated groups (Groups 2 or 3)

Descriptive statistics (mean, minimum, maximum, standard deviation, CV %, geometric mean and median) on flea counts for the various assessment days were calculated.

### Results

### Flea Counts

Arithmetic and geometric mean flea (Ctenocephalides felis) counts on the various assessment days for the three study groups are summarised below. The arithmetic mean flea count for the untreated control group (Group 1) ranged from 67.3 to 88.3 indicating vigorous flea challenges on all the assessment days. The geometric mean flea counts recorded for the IVP (PetArmor) and CVP (Frontline spot on dog) treated groups were statistically significantly less (p<0.05) than that of the untreated control group on all assessment days. No statistically significant differences (p>0.05) were recorded between the geometric mean flea counts recorded for the IVP (PetArmor) and CVP (Frontline Top Spot) treated groups on any of the assessment days.

| | | | GROUP 2 - IVP | | GROUP 3 - CVP | |
|---|---|---|---|---|---|---|
| | GROUP 1 - Negative | | (Fipronil for | | (Frontline | |
| | control | | dogs - PetArmor) | | Top Spot) | |
| | Arith- | Geo- | Arith- | Geo- | Arith- | Geo- |
| | metic | metric | metic | metric | metic | metric |
| DAY | mean | mean | mean | mean¹ | mean | mean² |
| +2 | 76.4 | 74.7 | 0.0 | 0.0 | 0.3 | 0.1 |
| +9 | 88.3 | 87.5 | 0.0 | 0.0 | 0.0 | 0.0 |
| +16 | 67.3 | 65.6 | 0.0 | 0.0 | 0.0 | 0.0 |
| +23 | 80.1 | 79.1 | 0.0 | 0.0 | 0.0 | 0.0 |
| +32 | 77.3 | 75.3 | 0.6 | 0.3 | 0.8 | 0.5 |

¹Group 2 differed statistically significant (p < 0.05) from the negative control Group 1
²Group 3 differed statistically significant (p < 0.05) from the negative control Group 1

### Efficacy Data

Efficacy values (%) based on arithmetic and geometric mean flea (Ctenocephalides felis) counts for the groups treated with the IVP and CVP are summarised below:

| | EFFICACIES (%) | | | |
|---|---|---|---|---|
| | GROUP 2 - IVP | | | |
| | (Fipronil for dogs - | | GROUP 3 - CVP | |
| | PetArmor) | | (Frontline spot-on dog) | |
| | Arithmetic | | Arithmetic | |
| DAY | mean | Geometric mean | mean | Geometric mean |
| +2 | 100.0 | 100.0 | 99.7 | 99.8 |
| +9 | 100 0 | 100.0 | 100.0 | 100.0 |
| +16 | 100.0 | 100.0 | 100.0 | 100.0 |
| +23 | 100.0 | 100.0 | 100.0 | 100.0 |
| +32 | 99.2 | 99.6 | 99.0 | 99.4 |

Efficacies based on geometric means were considered primary. Immediate efficacies (Day +2)>99% were recorded for both treated groups. Persistent efficacies (>99%) were recorded for both treatment groups up to 30 days post treatment.

### Conclusion

The IVP (PetArmor) and the CVP (Frontline Top Spot), administered to dogs at a dose rate of 0.067 ml/kg bodyweight, had similar immediate and persistent efficacies when challenged up to 30 days post treatment with Ctenocephalides felis.

### Comparative Efficacy Study—IV

### Objectives

To determine and compare the adulticidal efficacy against ticks (Rhipicephalus sanguineus and Dermacentor variabilis) of a fipronil spot-on formulation of the present invention to that of Frontline Top Spot for dogs, when administered to dogs.

### Study Design and Groups

This study was a parallel group design, randomised, unicentre, blinded controlled efficacy study. The study was conducted on three groups of eight dogs each.

Group 1: Untreated control
Group 2: Dogs were treated with the IVP (PetArmor) at a dosage of 0.067 ml/kg b.w.
Group 3: Dogs were treated with the CVP (Frontline) at a dosage of 0.067 ml/kg b.w.

### Randomisation

The study followed a randomised block design.

### Treatments

In this study the IVP and CVP were applied once at the beginning of the study (Day 0).

Treatments followed the dose level as set out below:

| Study | Sample | | Active | | Appli- | |
|---|---|---|---|---|---|---|
| group | size | IVP/CVP | ingredient | Dosages | cation | Day |
| 2 | 8 | PetArmor | Fipronil | 0.067 | Topical | 0 |
| | | | | ml/kg b.w. | spot-on | |
| 3 | 8 | Frontline | Fipronil | 0.067 | Topical | 0 |
| | | Top Spot | | ml/kg b.w. | spot-on | |

### Study Procedures

### Tick Infestations

Laboratory-bred strains of Rhipicephalus sanguineus and Dermacentor variabilis (Dermacentor reticulates on Day −1, see Deviation #3) were used in the artificial infestations. Immature ticks were fed on rabbits and adult ticks, which were used in the challenge infestations, were unfed, at least one week old and had a balanced sex ratio (50% female:50% male). Each dog was artificially infested with 50 ticks of each species on the days as set out in section 8.3. The time of infestation was recorded for all animals.

### Tick Counts

The times at which each animal was treated and at which it was infested with ticks were recorded. This was done to ensure that counting and removal of ticks were as close as possible to the specified target times (48±2 hour post infestation or treatment). The time of tick counting and removal was recorded. Ticks were found by direct observation following parting of the hair coat and palpation. Areas examined, not necessarily in this order, were:

Outside hind legs, including feet
Tail and anal areas
Lateral area, not including shoulders
Abdominal area, from chest to inside hind legs
Fore legs and shoulders, including feet
All neck and head areas
Dorsal strip from shoulder blades to base of tail

Ticks removed were recorded on the appropriate data capture form within sex according to the parameters given below:

| Category | General findings | Attachment status |
|---|---|---|
| 1 | Live | Free |
| 2 | Live | Attached; unengorged* |
| 3 | Live | Attached; engorged** |
| 4 | Killed | Free |
| 5 | Killed | Attached; unengorged* |
| 6 | Killed | Attached; engorged** |

*no filling of the alloscutum evident
**obvious or conspicuous filling of the alloscutum evident

All dogs were combed following the 48 hour tick counts and removal to ensure that all ticks were counted and removed.

### Statistical Methods

### Adulticidal Efficacy

The efficacy against ticks was calculated for the treatment groups at each assessment day according to the formulas given below. Due to the fact that small and even zero tick counts were recorded it was expected that the tick counts would not follow a normal distribution and so the primary efficacy calculations were based on geometric means rather than arithmetic means. The calculations were based on the geometric means of the tick (count+1) data and one (1) was subsequently subtracted from the result to obtain a meaningful value for the geometric mean of each group. Efficacy calculations based on arithmetic means were also included as part of the statistics package.

Efficacy against ticks was calculated according to the following formula:

Efficacy(%)=100×(m_{c}−mₜ)/m_{c}, where

m_{c}=Geometric/arithmetic mean number of live ticks (categories 1-3) on dogs in the negative control group (Group 1) at a specific time point.
mₜ=Geometric/arithmetic mean number of live and dead ticks (categories 1-3 & 6) on dogs in the treatment group (Groups 2 and 3) at a specific time point.

Descriptive statistics (mean, minimum, maximum, standard deviation, CV %, geometric mean and median) on tick counts for the various assessment days were calculated

### Results

### Tick Counts

Arithmetic and geometric mean tick (Rhipicephalus sanguineus) counts on the various assessment days for the three study groups are summarised below. The arithmetic mean tick count for the untreated control group (Group 1) ranged from 17.8 to 27.9 indicating vigorous tick challenges on all the assessment days. The geometric mean tick counts recorded for the IVP (PetArmor) and CVP (Frontline Top Spot for Dogs) treated groups were statistically significantly less (p<0.05) than those of the untreated control group on all assessment days. The geometric mean tick counts recorded for the IVP (PetArmor) treated group were statistically significantly less (p<0.05) than those recorded for the CVP (Frontline Top Spot for Dogs) treated group on Days +23 and +32.

| | | | GROUP 2 - IVP | | GROUP 3 - CVP | |
|---|---|---|---|---|---|---|
| | GROUP 1 - Negative | | (Fipronil for | | (Frontline Top | |
| | control | | dogs - PetArmor) | | Spot for Dogs) | |
| | Arith- | Geo- | Arith- | Geo- | Arith- | Geo- |
| | metic | metric | metic | metric | metic | metric |
| DAY | mean | mean | mean | mean¹ | mean | mean² |
| +2 | 27.9 | 23.1 | 4.9 | 4.0 | 11.1 | 7.2 |
| +9 | 19.8 | 16.6 | 0.6 | 0.4 | 0.6 | 0.5 |
| +16 | 22.9 | 21.2 | 1.3 | 0.8 | 2.4 | 2.0 |
| +23 | 23.5 | 21.3 | 0.4 | 0.3³ | 3.0 | 2.7 |
| +32 | 17.8 | 15.2 | 1.3 | 0.9³ | 5.5 | 3.5 |

¹Group 2 differed statistically significantly (p < 0.05 from the negative control Group 1
²Group 3 differed statistically significantly (p < 0.05 from the negative control Group 1
³Group 2 differed statistically significantly (p < 0.05 from Group 3

Arithmetic and geometric mean tick (Dermacentor variabilis and Dermacentor reticulatus) counts on the various assessment days for the three study groups are summarised below. The arithmetic mean tick count for the untreated control group (Group 1) ranged from 20.1 to 30.1 indicating vigorous tick challenges on all the assessment days. The geometric mean tick counts recorded for the IVP (PetArmor) and CVP (Frontline Top Spot for Dogs) treated groups were statistically significantly less (p<0.05) than that of the untreated control group on all assessment days. No statistically significant differences (p>0.05) were recorded on any of the assessment days between the geometric mean tick counts for the IVP (PetArmor) and CVP (Frontline Top Spot for Dogs) treated groups.

| | | | GROUP 2 - IVP | | GROUP 3 - CVP | |
|---|---|---|---|---|---|---|
| | GROUP 1 - Negative | | (Fipronil for | | (Frontline Top | |
| | control | | dogs - PetArmor) | | Spot for Dogs) | |
| | Arith- | Geo- | Arith- | Geo- | Arith- | Geo- |
| | metic | metric | metic | metric | metic | metric |
| DAY | mean | mean | mean | mean¹ | mean | mean² |
| +2* | 28.1 | 22.2 | 4.8 | 3.8 | 9.6 | 5.1 |
| +9 | 20.8 | 16.7 | 0.4 | 0.3 | 0.0 | 0.0 |
| +16 | 22.3 | 20.9 | 0.0 | 0.0 | 0.3 | 0.1 |
| +23 | 30.1 | 28.5 | 0.4 | 0.3 | 1.4 | 1.0 |
| +32 | 20.1 | 18.9 | 0.1 | 0.1 | 0.5 | 0.3 |

*Dermacentor reticulatus
¹Group 2 differed statistically significantly (p < 0.05 from the negative control Group 1
²Group 3 differed statistically significantly (p < 0.05 from the negative control Group 1

### Efficacy Data

Efficacy values (%) based on arithmetic and geometric mean tick (Rhipicephalus sanguineus) counts for the groups treated with the IVP and CVP are summarised below:

| | EFFICACIES (%) | | | |
|---|---|---|---|---|
| | GROUP 2 - IVP | | | |
| | (Fipronil for dogs - | | GROUP 3 - CVP | |
| | PetArmor) | | (Frontline Top Spot for Dogs) | |
| | Arithmetic | | Arithmetic | |
| DAY | mean | Geometric mean¹ | mean | Geometric mean |
| +2 | 82.5 | 82.5 | 60.1 | 68.8 |
| +9 | 96.8 | 97.8 | 96.8 | 96.7 |
| +16 | 94.5 | 96.3 | 89.6 | 90.8 |
| +23 | 98.4 | 98.8 | 87.2 | 87.1 |
| +32 | 93.0 | 94.2 | 69.0 | 77.2 |

Efficacies based on geometric means were considered primary. No immediate efficacies (Day +2)>90% were recorded for the IVP (PetArmor) or CVP (Frontline Top Spot for Dogs) treated groups. The IVP (PetArmor) had however a markedly higher immediate (Day +2) efficacy compared to that of the CVP (Frontline Top Spot for Dogs). Persistent efficacies (>90%) were recorded for the IVP (PetArmor) treated group up to Day +32 and for the CVP (Frontline Top Spot for Dogs) treatment group up to Day +16.

Efficacy values (%) based on arithmetic and geometric mean tick (Dermacentor reticulatus and Dermacentor variabilis) counts for the groups treated with the IVP and CVP are summarised below:

| | EFFICACIES (%) | | | |
|---|---|---|---|---|
| | GROUP 2 - IVP | | | |
| | (Fipronil for dogs - | | GROUP 3 - CVP | |
| | PetArmor) | | (Frontline Top Spot for Dogs) | |
| | Arithmetic | | Arithmetic | |
| DAY | mean | Geometric mean¹ | mean | Geometric mean |
| +2 | 83.1 | 82.9 | 65.8 | 76.9 |
| +9 | 98.2 | 98.5 | 100.0 | 100.0 |
| +16 | 100.0 | 100.0 | 98.9 | 99.3 |
| +23 | 98.8 | 99.0 | 95.4 | 96.5 |
| +32 | 99.4 | 99.5 | 97.5 | 98.3 |

Dermacentor reticulatus

Efficacies based on geometric means were considered primary. No immediate efficacies (Day +2)>90% were recorded for the IVP (PetArmor) or CVP (Frontline Top Spot for Dogs) treated groups. Persistent efficacies (>90%) were recorded for the IVP (PetArmor) and CVP (Frontline Top Spot for Dogs) treatment groups up to 32 days post treatment.

### Conclusion

The IVP (PetArmor) had a markedly greater immediate efficacy (82.5%) than the CVP (Frontline Top Spot for Dogs) which was 68.8% against Rhipicephalus sanguineus ticks on dogs when assessed 48 h after treatment. The IVP (PetArmor) was also persistently more effective (>90%) than the CVP (Frontline Top Spot for Dogs) in treating dogs infested with Rhipicephalus sanguineus ticks up to 32 days after treatment with significant differences between the geometric mean burdens on Days 23 and 32.

The IVP (PetArmor) had a greater immediate efficacy (82.9%) than the CVP (Frontline Top Spot for Dogs) (76.9%) against Dermacentor reticulatus ticks on dogs when assessed 48 h after treatment.

The IVP (PetArmor) and CVP (Frontline Top Spot for Dogs) had similar persistent efficacies (>90%) up to 32 days post treatment against Dermacentor variabilis tick infestations.

### Comparative Efficacy Study—V

### Objectives

To determine and compare the adulticidal efficacy against ticks (Dermacentor variabilis) of a fipronil spot-on formulation of the present invention to that of Frontline Top Spot for dogs, when administered to cats.

### Study Design and Groups

This study was a parallel group design, randomised, unicentre, blinded controlled efficacy study. In order to control bias, the animals were treated by an individual not involved in performing the post-treatment assessments and observations. Study groups were coded to blind the post-treatment observations and assessments.

The study was conducted on three groups of eight cats each.

Group 1: Untreated control
Group 2: Cats were treated with the IVP (PetArmor) at a dosage of 0.5 ml/cat
Group 3: Cats were treated with the CVP (Frontline) at a dosage of 0.5 ml/cat

### Randomisation

The study followed a randomised block design.

### Treatments

In this study the IVP and CVP was applied once at the beginning of the study (Day 0). Treatments followed the dose level as set out below:

| Study | Sample | | Active | | Appli- | |
|---|---|---|---|---|---|---|
| group | size | IVP/CVP | ingredient | Dosages | cation | Day |
| 2 | 8 | PetArmor | Fipronil | 0.5 | Topical | 0 |
| | | | | ml/cat | spot-on | |
| 3 | 8 | Frontline Top | Fipronil | 0.5 | Topical | 0 |
| | | Spot cat | | ml/cat | spot-on | |

### Study Procedures

### Tick Infestations

A laboratory-bred strain of Dermacentor variabilis (US Oklahoma strain) ticks was used in the artificial infestations. Immature ticks were fed on rabbits. Adult ticks, which were used in the artificial infestations, were unfed, at least one week old and had a balanced sex ratio (50% female:50% male). Each cat was artificially infested (whole body infestation) with 50 ticks on Days −6, −1, +7, +14, +21 and +30. Cats were sedated to allow infestation. The ticks were not placed on or near the site of IVP/CVP application after treatment. The time of infestation was recorded for all animals. Immediately following infestation the cats were fitted with a collar to prevent grooming.

### Tick Counts

The time at which each animal was treated or at which it was infested with ticks was recorded.

This was done to ensure that counting and removal of ticks were as close as possible to the specified target times (48±2 hr post infestation or treatment). The time of tick counting and removal was recorded. Ticks were found by direct observation following parting of the hair coat and palpation. Areas examined, not necessarily in this order, were:

Outside hind legs, including feet
Tail and anal areas
Lateral area, not including shoulders
Abdominal area, from chest to inside hind legs
Fore legs and shoulders, including feet
All neck and head areas
Dorsal strip from shoulder blades to base of tail

Ticks removed were categorized and recorded according to the parameters given below:

| Category | General findings | Attachment status |
|---|---|---|
| 1 | Live | Free |
| 2 | Live | Attached; unengorged* |
| 3 | Live | Attached; engorged** |
| 4 | Killed | Free |
| 5 | Killed | Attached; unengorged* |
| 6 | Killed | Attached; engorged** |

*no filling of the alloscutum evident
**obvious or conspicuous filling of the alloscutum evident

### Statistical Methods

### Adulticidal Efficacy

The efficacy against ticks was calculated for each treatment group at each assessment day according to the formulas given below. Due to the fact that small and even zero tick counts were recorded it was expected that the tick counts would not follow a normal distribution. It was therefore decided that the primary efficacy calculations would be based on geometric means rather than arithmetic means. The calculations were based on the geometric means of the tick (count+1) data. One (1) was subsequently subtracted from the result to obtain a meaningful value for the geometric mean of each group. Efficacy calculations based on arithmetic means were also calculated.

Percent efficacy for the treated group and day against ticks were calculated as follows:

Efficacy(%)against ticks=100×(Gm_{c}−Gmₜ)/Gm_{c}, where:

Gm_{c}=Geometric or arithmetic mean number of live ticks (categories 1-3) on cats in the negative control group (Group 1) at a specific time point.
Gmₜ=Geometric or arithmetic mean number of live ticks (categories 1-3; immediate efficacy) and live and dead attached engorged ticks (categories 1-3 & 6; persistent efficacies) on cats in the treatment group (Groups 2 and 3) at a specific time point.

Descriptive statistics (mean, minimum, maximum, standard deviation, CV %, geometric mean and median) on tick counts for the various assessment days were calculated.

### Results

### Tick Counts

Arithmetic and geometric mean Dermacentor variabilis counts on the various assessment days for the three study groups are summarised below. The arithmetic mean tick counts recorded for the untreated control Group 1 ranged from 11.8 to 25.0, indicating vigorous tick challenges on all post treatment assessment days. The geometric mean tick counts recorded for the IVP (PetArmor) and CVP (Frontline Top Spot cat) treated groups were statistically significantly (p<0.05) less than that recorded for the untreated control Group 1 from one to four weeks post treatment. No statistically significant differences (p>0.05) in geometric mean tick counts were observed between the IVP treated Group 2 (PetArmor) and the CVP treated Group 3 (Frontline Top Spot cat) on any of the assessment days.

| | | | | | GROUP 3 - CVP | |
|---|---|---|---|---|---|---|
| | GROUP 1 - Negative | | GROUP 2 - IVP | | (Frontline | |
| | control | | (PetArmor) | | Top Spot cats) | |
| | Arith- | Geo- | Arith- | Geo- | Arith- | Geo- |
| | metic | metric | metic | metric | metic | metric |
| DAY | mean | mean | mean | mean | mean | mean² |
| +2 | 11.8 | 9.6 | 11.1 | 7.9 | 7.5 | 4.1 |
| +9 | 22.1 | 20.6 | 1.1 | 0.8¹ | 2.4 | 1.0¹ |
| +16 | 22.9 | 21.6 | 1.5 | 0.9¹ | 2.1 | 1.2¹ |
| +23 | 15.4 | 14.4 | 3.3 | 2.1¹ | 3.1 | 2.4¹ |
| +32 | 25.0 | 24.7 | 9.0 | 7.3¹ | 9.9 | 8.4¹ |

¹Group 2 and Group 3 differed statistically significantly (p < 0.05) from the untreated control Group 1

### Efficacy Data

Efficacy values (%) based on arithmetic and geometric means for the groups treated with the IVP and CVP against Dermacentor variabilis ticks are summarised below:

| | EFFICACIES (%) | | | |
|---|---|---|---|---|
| | GROUP 2 - IVP | | GROUP 3 - CVP | |
| | (PetArmor) | | (Frontline Top Spot cat) | |
| | Arithmetic | | Arithmetic | |
| DAY | mean | Geometric mean | mean | Geometric mean² |
| +2 | 5.3 | 17.4 | 36.2 | 57.6 |
| +9 | 94.9 | 96.3 | 89.3 | 95.2 |
| +16 | 93.4 | 96.0 | 90.7 | 94.2 |
| +23 | 78.9 | 85.7 | 79.7 | 83.0 |
| +32 | 64.0 | 70.3 | 60.5 | 65.9 |

Efficacies based on geometric means were considered primary. No immediate efficacies (Day +2)>90% were recorded for the IVP (PetArmor) or CVP (Frontline Top Spot cat) treated groups. The CVP (Frontline Top Spot cat) had a markedly higher immediate (Day +2) efficacy compared to that of the IVP (PetArmor). Both the IVP (PetArmor) and CVP (Frontline Top Spot cat) had >90% persistent efficacies up to Day +16. Comparable persistent efficacies were recorded for the IVP (PetArmor) and CVP (Frontline Top Spot cat) treated groups up to Day +32.

### Conclusion

Both the IVP (PetArmor) and CVP (Frontline Top Spot cat) had immediate efficacies well below 90%. Although not clinically significant, the CVP (Frontline Top Spot cat) had a greater immediate efficacy (57.6%) than the IVP (PetArmor) against Dermacentor variabilis ticks on cats when challenged on the day before treatment and assessed 48 h after treatment. Both the IVP (PetArmor) and CVP (Frontline Top Spot cat) had >90% persistent efficacies up to Day +16 and comparable persistent efficacies up to Day +32.

### Comparative Efficacy Study—VI

### Objectives

To determine and compare the adulticidal efficacy against fleas (Ctenocephalides felis) of a fipronil spot-on formulation of the present invention to that of Frontline Top Spot cat, when administered to cats.

### Study Design and Groups

This study was a parallel group design, randomised, unicentre, blinded controlled efficacy study.

The study was conducted on three groups of eight cats each.

Group 1: Untreated control
Group 2: Cats treated with the IVP (PetArmor) at a dosage of 0.5 ml/cat
Group 3: Cats treated with the CVP (Frontline) at a dosage of 0.5 ml/cat

### Randomisation

The study followed a randomised block design.

### Treatments

In this study the IVP and CVP were applied once at the beginning of the study (Day 0).

Treatments followed the dose level as set out below:

| Study | Sample | | Active | | Appli- | |
|---|---|---|---|---|---|---|
| group | size | IVP/CVP | ingredient | Dosages | cation | Day |
| 2 | 8 | PetArmor | Fipronil | 0.5 | Topical | 0 |
| | | | | ml/cat | spot-on | |
| 3 | 8 | Frontline Top | Fipronil | 0.5 | Topical | 0 |
| | | Spot cat | | ml/cat | spot-on | |

### Study Procedures

### Flea Infestations

A laboratory bred strain (PLRS US strain) of Ctenocephalides felis (routinely fed on cats) was used for all infestations. Fleas were unfed and of mixed sex. Each cat was infested with 100 fleas on Days −6, −1, +7, +14, +21 and +30. The fleas were not placed on or near the site of IVP/CVP application after treatment. The time of infestation was recorded for all animals.

### Flea Counts

Body flea counts were conducted as close as possible to the specified target times (48±2 hr post-treatment or infestation) on Days −4, +2, +9, +16, +23 and +32. The time of flea counting was recorded. During combing a fine-toothed flea comb was used to recover fleas present in the animal's fur. The method of combing was by several strokes of the comb in each area of the animal, each time moving in the same direction following the pattern of the hair coat. Movement, from one part of the animal's fur to the next was via strokes overlapping each other, so that no area of fur was missed. Areas examined, not necessarily in this order, were:

Outside hind legs, including feet
Tail and anal areas
Lateral area, not including shoulders
Abdominal area, from chest to inside hind legs
Forelegs and shoulders, including feet
All neck and head areas
Dorsal strip from shoulder blades to base of tail

After completion of the combing procedure for all body areas, the whole procedure was repeated once more so that all areas were combed a minimum of two times. When necessary, the combing procedure was continued for a third time or more until no live fleas were found.

### Statistical Methods

### Adulticidal Efficacy

The efficacy against fleas was calculated for the treatment groups at each assessment day according to the formulas given below. Due to the fact that small and even zero flea counts were recorded it was expected that the flea counts would not follow a normal distribution and so the primary efficacy calculations were based on geometric means rather than arithmetic means. The calculations were based on the geometric means of the flea (count+1) data and one (1) was subsequently subtracted from the result to obtain a meaningful value for the geometric mean of each group. Efficacy calculations based on arithmetic means were also included as part of the statistics package.

Efficacy against fleas were calculated according to the following formula:

Efficacy(%)=100×(m_{c}−mₜ)/m_{c}, where

m_{c}=geometric/arithmetic mean of live fleas on the negative control group (Group 1)
mₜ=geometric/arithmetic mean of live fleas on the IVP/CVP treated groups (Groups 2 or 3)

Descriptive statistics (mean, minimum, maximum, standard deviation, CV %, geometric mean and median) on flea counts for the various assessment days were calculated.

### Results

### Flea Counts

Arithmetic and geometric mean flea (Ctenocephalides felis) counts on the various assessment days for the three study groups are summarised below. The arithmetic mean flea count for the untreated control group (Group 1) ranged from 50.6 to 57.4 indicating vigorous flea challenges on all the assessment days. The geometric mean flea counts recorded for the IVP (PetArmor) and CVP (Frontline spot cat) treated groups were statistically significantly less (p<0.05) than that of the untreated control group on all assessment days. No statistically significant differences (p>0.05) were recorded between the geometric mean flea counts recorded for the IVP (PetArmor) and CVP (Frontline Top Spot) treated groups.

| | | | GROUP 2 - IVP | | GROUP 3 - CVP | |
|---|---|---|---|---|---|---|
| | GROUP 1 - Negative | | (Fipronil for | | (Frontline | |
| | control | | cats - PetArmor) | | top spot cat) | |
| | Arith- | Geo- | Arith- | Geo- | Arith- | Geo- |
| | metic | metric | metic | metric | metic | metric |
| DAY | mean | mean | mean | mean¹ | mean | mean² |
| +2 | 50.6 | 50.4 | 0.1 | 0.1 | 0.1 | 0.1 |
| +9 | 55.4 | 54.9 | 0.0 | 0.0 | 0.0 | 0.0 |
| +16 | 57.4 | 56.2 | 0.0 | 0.0 | 0.0 | 0.0 |
| +23 | 51.6 | 48.7 | 0.0 | 0.0 | 0.0 | 0.0 |
| +32 | 57.4 | 57.1 | 0.0 | 0.0 | 0.3 | 0.1 |

¹Group 2 differed statistically significantly (p < 0.05) from the negative control Group 1
²Group 3 differed statistically significantly (p < 0.05) from the negative control Group 1

### Efficacy Data

Efficacy values (%) based on arithmetic and geometric mean flea (Ctenocephalides felis) counts for the groups treated with the IVP and CVP are summarised below:

| | EFFICACIES (%) | | | |
|---|---|---|---|---|
| | GROUP 2 - IVP | | GROUP 3 - CVP | |
| | (Fipronil for cats - PetArmor) | | (Frontline top spot cat) | |
| | Arithmetic | | Arithmetic | |
| DAY | mean | Geometric mean | mean | Geometric mean |
| +2 | 99.8 | 99.8 | 99.8 | 99.8 |
| +9 | 100.0 | 100.0 | 100.0 | 100.0 |
| +16 | 100.0 | 100.0 | 100.0 | 100.0 |
| +23 | 100.0 | 100.0 | 100.0 | 100.0 |
| +32 | 100.0 | 100.0 | 99.6 | 99.7 |

Efficacies based on geometric means were considered primary. Immediate efficacies (Day +2)>99% were recorded for both IVP (PetArmor) and CVP (Frontline top spot cat) treated groups. Persistent efficacies (>99%) were recorded for both IVP (PetArmor) and CVP (Frontline top spot cat) treated groups up to 32 days post treatment.

### Conclusion

The IVP (PetArmor) and the CVP (Frontline Top Spot), administered to cats at a dose rate of 0.5 ml/cat, had similar immediate and persistent efficacies against challenge with Ctenocephalides felis up to 30 days post treatment.

All publications and patent applications herein are incorporated by reference to the same extent as if each individual publication or patent application was specifically and individually indicated to be incorporated by reference.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A parasiticidal spot-on formulation comprising
fipronil or a veterinary acceptable salt thereof;
an insect growth regulator or veterinary acceptable salt thereof;
a pyrethroid or a veterinary acceptable salt thereof comprising less than about 40% by weight of the formulation; and
an organic solvent.

2. The formulation of claim 1 wherein the fipronil or veterinary acceptable salt thereof is present at an amount of about 5 to about 12% by weight of the formulation.

3. The formulation of claim 1 wherein the insect growth regulator is s-methoprene.

4. The formulation of claim 3 wherein the s-methoprene is present in an amount of about 7 to about 12% of the formulation.

5. The formulation of claim 1 wherein the pyrethroid is permethrin.

6. The formulation of claim 1, comprising a C₁-C₆ alcohol co-solvent

7. The formulation of claim 6 wherein the C₁-C₆ alcohol co-solvent is selected from the group consisting of ethanol, propanol, isopropanol, and a combination thereof.

8. The formulation of claim 1 comprising one or more antioxidants.

9. The formulation of claim 8, wherein the one or more antioxidants are selected from the group consists of butylated hydroxylanisole (BHA), butylated hydroxyltoluene (BHT), and α-Tocopherol.

10. The formulation of claim 8, wherein the one or more antioxidants are butylated hydroxylanisole (BHA) and butylated hydroxyltoluene (BHT).

11. The formulation of claim 8 wherein the one or more antioxidants are butylated hydroxylanisole (BHA), butylated hydroxyltoluene (BHT), and α-Tocopherol.

12. The formulation of claim 1, wherein the at least one organic solvent is diethylene glycol monoethyl ether.

13. The formulation of claim 5 further comprising a potentiator of permethrin.

14. The formulation of claim 13 wherein the potentiator of permethrin is piperonyl butoxide.

15. A parasiticidal spot-on formulation comprising
fipronil or a veterinary acceptable salt thereof;
a juvenile hormone analogue;
a pyrethroid or a veterinary acceptable salt thereof comprising less than about 40% by weight of the formulation; and
an organic solvent.

16. The formulation of claim 15 wherein the fipronil or veterinary acceptable salt thereof is present at an amount of about 5 to about 12% of the formulation.

17. The formulation of claim 15 wherein the juvenile hormone analogue is pyriproxifen.

18. The formulation of claim 17 wherein the pyriproxifen is present at about 1 to about 10% of the formulation.

19. The formulation of claim 15 wherein the pyrethroid is permethrin.

20. The formulation of claim 15, comprising a C₁-C₆ alcohol co-solvent

21. The formulation of claim 20 wherein the C₁-C₆ alcohol co-solvent is selected from the group consisting of ethanol, propanol, isopropanol, and a combination thereof.

22. The formulation of claim 15 comprising one or more antioxidants.

23. The formulation of claim 22, wherein the one or more antioxidants are selected from the group consists of butylated hydroxylanisole (BHA), butylated hydroxyltoluene (BHT), and α-Tocopherol.

24. The formulation of claim 22, wherein the one or more antioxidants are butylated hydroxylanisole (BHA) and butylated hydroxyltoluene (BHT).

25. The formulation of claim 22, wherein the one or more antioxidants are butylated hydroxylanisole (BHA), butylated hydroxyltoluene (BHT), and α-Tocopherol.

26. The formulation of claim 15, wherein the at least one organic solvent is diethylene glycol monoethyl ether.

27. The formulation of claim 19 further comprising a potentiator of permethrin.

28. The formulation of claim 27 wherein the potentiator of permethrin is piperonyl butoxide.

29. A parasiticidal spot-on formulation comprising
fipronil or a veterinary acceptable salt thereof;
an insect growth regulator;
a pyrethroid or a veterinary acceptable salt thereof comprising less than about 20% by weight of the formulation; and
an organic solvent.

30. The formulation of claim 29 wherein the fipronil or veterinary acceptable salt thereof is present at an amount of about 5 to about 12% of the formulation.

31. The formulation of claim 29 wherein the insect growth regulator is s-methoprene.

32. The formulation of claim 31 wherein the s-methoprene is present in an amount of about 7 to about 12% of the formulation.

33. The formulation of claim 29 wherein the pyrethroid is permethrin.

34. The formulation of claim 29, comprising a C₁-C₆ alcohol co-solvent

35. The formulation of claim 34 wherein the C₁-C₆ alcohol co-solvent is selected from the group consisting of ethanol, propanol, isopropanol, and a combination thereof.

36. The formulation of claim 29 comprising one or more antioxidants.

37. The formulation of claim 36, wherein the one or more antioxidants are selected from the group consists of butylated hydroxylanisole (BHA), butylated hydroxyltoluene (BHT), and α-Tocopherol.

38. The formulation of claim 36, wherein the one or more antioxidants are butylated hydroxylanisole (BHA) and butylated hydroxyltoluene (BHT).

39. The formulation of claim 36 wherein the one or more antioxidants are butylated hydroxylanisole (BHA), butylated hydroxyltoluene (BHT), and α-Tocopherol.

40. The formulation of claim 29, wherein the at least one organic solvent is diethylene glycol monoethyl ether.

41. The formulation of claim 33 further comprising a potentiator of permethrin.

42. The formulation of claim 41 wherein the potentiator of permethrin is piperonyl butoxide.

43. A parasiticidal spot-on formulation comprising
fipronil or a veterinary acceptable salt thereof, which is present from about 5% to about 15% by weight of the formulation;
a juvenile hormone analogue at about 1% to about 10% by weight of the formulation;
a pyrethroid or a veterinary acceptable salt thereof comprising less than about 40% by weight of the formulation; and
an organic solvent.

44. The formulation of claim 43 wherein the fipronil or veterinary acceptable salt thereof is present at an amount of about 5 to about 12% of the formulation.

45. The formulation of claim 43 wherein the juvenile hormone analogue is pyriproxifen.

46. The formulation of claim 45 wherein the pyriproxifen is present at an about of about 1 to about 10% of the formulation.

47. The formulation of claim 43 wherein the pyrethroid is permethrin.

48. The formulation of claim 43, comprising a C₁-C₆ alcohol co-solvent

49. The formulation of claim 48 wherein the C₁-C₆ alcohol co-solvent is selected from the group consisting of ethanol, propanol, isopropanol, and a combination thereof.

50. The formulation of claim 43 comprising one or more antioxidants.

51. The formulation of claim 50, wherein the one or more antioxidants are selected from the group consists of butylated hydroxylanisole (BHA), butylated hydroxyltoluene (BHT), and α-Tocopherol.

52. The formulation of claim 50, wherein the one or more antioxidants are butylated hydroxylanisole (BHA) and butylated hydroxyltoluene (BHT).

53. The formulation of claim 50 wherein the one or more antioxidants are butylated hydroxylanisole (BHA), butylated hydroxyltoluene (BHT), and α-Tocopherol.

54. The formulation of claim 37, wherein the at least one organic solvent is diethylene glycol monoethyl ether.

55. The formulation of claim 47 further comprising a potentiator of permethrin.

56. The formulation of claim 55 wherein the potentiator of permethrin is piperonyl butoxide.
